(19) United States
(12) Patent Application Publication
(10) Pub. No.: US 2007/0293814 A1
(43) Pub. Date: Dec. 20, 2007

(54) Coatable transdermal delivery microprojection assembly

(76) Inventors: Joseph C. Trautman, Sunnyvale, CA (US); Ling-Kang Tong, Fremont, CA (US); Doran P. Donnelly, Redwood City, CA (US)

Correspondence Address: WILSON SONSINI GOODRICH & ROSATI & MACROFLUX CORP., 650 PAGE MILL ROAD, PALO ALTO, CA, 94304, US

(21) Appl. No.: 11/518,970

(22) Filed: Sep. 11, 2006

Related U.S. Application Data
(60) Provisional application No. 60/716,459, filed on Sep. 12, 2005.

(51) Int. Cl. A61M 37/00

(52) U.S. Cl. 604/46

(57) ABSTRACT

The present invention is a transdermal delivery microprojection assembly that includes a microprojection member secured to a retainer adapted for use with an impact applicator wherein at least a portion of the microprojections extend beyond a plane formed by the end of the retainer. The configuration allows a biocompatible coating containing a biologically active agent to be applied to the microprojection member after it is mounted on the retainer. The present invention minimizes the number of manufacturing steps that must be carried out under aseptic conditions to maintain sterility of the assembly after the coating is applied to the microprojection member.

### CROSS REFERENCE TO RELATED APPLICATIONS

This application claims the benefit of U.S. Provisional Application Ser. No. 60/716,459, filed Sep. 12, 2005.

### FIELD OF THE PRESENT INVENTION

The invention relates generally to transdermal delivery systems. More particularly, the invention relates to a microprojection member assembly adapted to penetrate the skin that can be readily coated with a biologically active agent.

### BACKGROUND OF THE INVENTION

As is well known in the art, transdermal delivery provides for a method of administering active agents that would otherwise need to be delivered via hypodermic injection or intravenous infusion. Transdermal agent delivery offers improvements in both of these areas. Transdermal delivery, when compared to oral delivery, avoids the harsh environment of the digestive tract, bypasses gastrointestinal drug metabolism, reduces first-pass effects and avoids the possible deactivation by digestive and liver enzymes.

The word “transdermal”, as used herein, refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle.

As is also well known in the art, transdermal agent flux is dependent upon the condition of the skin, the size and physical/chemical properties of the agent molecule, and the concentration gradient across the skin. Because of the low permeability of the skin to many active agents, transdermal delivery has had limited applications. This low permeability is attributed primarily to the stratum corneum, the outermost skin layer, which consists of flat, dead cells filled with keratin fibers (i.e., keratinocytes) surrounded by lipid bilayers. This highly-ordered structure of the lipid bilayers confers a relatively impermeable character to the stratum corneum.

To increase transdermal diffusional agent flux, many techniques and systems have been developed to mechanically penetrate or disrupt the outermost skin layers thereby creating pathways into the skin in order to enhance the amount of agent being transdermally delivered. Early vaccination devices, known as scarifiers, generally included a plurality of tines or needles that were applied to the skin to and scratch or make small cuts in the area of application. The vaccine was applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

Other systems and apparatus employ tiny skin piercing elements to enhance transdermal drug delivery. Illustrative are the systems and apparatus disclosed in U.S. Pat. Nos. 5,879,326, 3,814,097, 5,279,54, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes, sizes and arrays to pierce the outermost layer (i.e., the stratum corneum) of the skin. The piercing elements in some of these devices are extremely small, some having a microprojection length of only about 25-400 microns and a microprojection thickness of only about 5-50 microns.

As disclosed in U.S. patent application Ser. No. 10/045,842, which is fully incorporated by reference herein, a biologically active agent that is to be delivered can be coated on the microprojections or microprojection array. This eliminates the necessity of a separate physical reservoir and developing an agent formulation or composition specifically for the reservoir.

When microprojection arrays are used to deliver a biologically active agent through the skin, consistent, complete, and repeatable penetration is desired. Manual application of a microprojection array often results in significant variation in puncture depth across the length and width of the array. In addition, manual application can result in large variations in puncture depth between applications, leading to inconsistent delivery amounts of the agent.

To overcome these and other deficiencies of manual application, an automatic applicator can be used to cause the microprojections to pierce the stratum consistently over the length and width of the microprojection array in a highly reproducible manner. For example, U.S. Pat. No. 6,855,131, which is hereby fully incorporated by reference, discloses a spring loaded applicator adapted to apply a microprojection array by impacting the array against the patient's skin. The microprojection array is mounted within a retainer ring that is adapted to mate with the applicator. The retainer ring allows the microprojection array to be mounted on the applicator without the need for the operator to touch the array.

An important consideration in any transdermal delivery system is achieving an appropriate level of sterility to meet the relevant bioburden specifications. Although sterilizing the microprojection array and retainer is relatively easy, sterilization of the microprojection array after it has been coated with a biologically active agent can be complicated and may lead to degradation of the agent. The use of aseptic manufacturing conditions following coating avoids the difficulties of terminal sterilization. Accordingly, it would be desirable to apply the biologically active agent coating after the microprojection array and retainer ring are assembled to minimize the number of manufacturing steps that must be carried out under aseptic conditions.

However, the retainer ring disclosed in the '131 patent places the microprojection array in a recessed position. This placement makes it very difficult to coat the microprojection array with the biologically active agent after it is mounted in the retainer. It would thus be desirable to provide a microprojection array and retainer assembly that facilitates coating the microprojection array after it is mounted on the retainer.

It is therefore an object of the present invention to provide a microprojection member or array and retainer assembly that substantially reduces or eliminates the aforementioned drawbacks and disadvantages associated with prior art microprojection devices.

It is another object of the present invention to provide a transdermal delivery assembly having a microprojection member that can be coated with a biologically active agent after the microprojection member is mounted on a retainer.

It is another object of the present invention to provide a transdermal delivery device that minimizes the number of manufacturing steps required after a coating having a biologically active agent is applied to the microprojection member.

### SUMMARY OF THE INVENTION

In accordance with the above objects and those that will be mentioned and will become apparent below, a transdermal delivery assembly of the present invention generally includes a microprojection member having top and bottom surfaces and a plurality of stratum corneum-piercing microprojections that project from the bottom surface of the microprojection member, and a retainer having first and second ends and a central opening, wherein the microprojection member is secured to the retainer within the central opening and wherein the microprojection member is positioned adjacent the first end of the retainer so that at least a portion of the microprojections extend beyond a plane formed by the first end of the retainer.

Preferably, the assembly also comprises an adhesive patch, wherein the microprojection member is secured to the patch and the patch is secured to the retainer.

In one embodiment, the patch is secured to the retainer by frangible tabs.

In one embodiment of the present invention, the patch has first and second sides and the microprojection member is secured to the first side. In the noted embodiment, the same adhesive used to secure the microprojection member and to adhere to the patient's skin is used to secure the patch to the retainer. Alternatively, the patch is secured to the retainer by a separate adhesive on the second side.

In one embodiment of the present invention, the first end of the retainer is configured to nest with the second end of the retainer so that a plurality of retainers having mounted microprojection members can be stacked. Preferably, the microprojection member is secured to the retainer so that the microprojection member does not contact adjacent microprojection members and adhesive patches when a plurality of assemblies are stacked.

In accordance with a further embodiment of the present invention, the transdermal delivery assembly also includes a housing having first and second ends and a central opening, wherein the housing is adapted to receive and position the retainer within the central opening of the housing and wherein the retainer is disposed within the housing. Preferably, the first end of the housing is adapted to releasably attach to an impact applicator. Also preferably, the retainer is positioned within the housing so that the microprojection member is spaced away from the first and second ends of the housing.

Preferably, the microprojection member is coated with an agent formulation that includes at least one biologically active agent.

In one embodiment, the biologically active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones, hGH, HMG, desmopressin acetate, follicle luteoids, aANF, growth factors, growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), amylin, insulinotropin, GLIP1, granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs, ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, alpha MSH, VEGF, PYY and hBNP.

In another embodiment of the invention, the active agent is an immunologically active agent selected from the group consisting of proteins, polysaccharide conjugates, oligosaccharides, lipoproteins, tetanus toxoid, diphtheria toxoid, botulinum toxoid, hemaglutinins, hepatitis B surface antigen, Bordetella pertussis (recombinant PT accince-acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

In a further embodiment of the invention, the biocompatible coating includes at least one additional pharmaceutical agent selected from the group consisting of pathway patency modulators and vasoconstrictors.

In accordance with another embodiment, the present invention is a method for producing a transdermal delivery assembly, including the steps of i) providing a microprojection member having top and bottom surfaces and a plurality of stratum corneum-piercing microprojections that project from the bottom surface of the microprojection member; ii) providing a retainer having first and second ends and a central opening; and iii) securing the microprojection member to the retainer within the central opening to form the transdermal delivery assembly wherein the microprojection member is positioned adjacent the first end of the retainer so that at least a portion of the microprojections extend beyond a plane formed by the first end of the retainer.

In one embodiment of the present invention, the method also includes the step of providing an adhesive patch and the step of securing the microprojection member to the retainer comprises securing the microprojection member to the patch and securing the patch to the retainer.

In another embodiment of the present invention, the method includes the step of sterilizing the transdermal delivery assembly.

In yet another embodiment of the invention, a biocompatible coating containing at least one biologically active agent is applied to the microprojection member after the transdermal delivery assembly is sterilized. Preferably, the biocompatible coating is applied to the microprojection member by roller coating. Alternatively, the biocompatible coating is applied to the microprojection member by dip-coating.

In accordance with another aspect of the present invention, the noted method also includes the steps of i) providing a housing having first and second ends and a central opening, wherein the housing is adapted to receive and position the retainer within the central opening of the housing and ii) placing the retainer within the housing after applying the biocompatible coating.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a front cross-sectional view of a prior art retainer;

FIG. 2 is a perspective view of the retainer shown in FIG. 2;

FIG. 3 is an exploded view of a microprojection member assembly, according to the invention;

FIG. 4 is a perspective view of the microprojection member assembly shown in FIG. 3;

FIG. 5 is an exploded view of an alternate microprojection member assembly, according to the invention;

FIG. 6 is a perspective view of the microprojection member assembly shown in FIG. 5;

FIG. 7 is a schematic view of the microprojection member assembly shown in FIG. 4 being coated with a roller, according to the invention;

FIG. 8 is a perspective view illustrating microprojection member assemblies of the type shown in FIG. 6 in a stacked configuration, according to the invention;

FIG. 9 is an exploded view of the microprojection member assembly of the type shown in FIG. 4 also including a housing, according to the invention;

FIG. 10 is a perspective view of the microprojection member assembly shown in FIG. 7; and

FIG. 11 is a perspective view of a portion of one example of a microprojection member, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a peptide” includes two or more such peptides; reference to “a microprojection” includes two or more such microprojections and the like.

### Definitions

The term “transdermal”, as used herein, means the delivery of an agent into and/or through the skin for local or systemic therapy. The term “transdermal” thus means and includes intracutaneous, intradermal and intraepidermal delivery of an agent, such as a peptide, into and/or through the skin via passive diffusion as well as energy-based diffusional delivery, such as iontophoresis and phonophoresis.

The term “transdermal flux”, as used herein, means the rate of transdermal delivery.

The term “active agent”, as used herein, refers to a composition of matter or mixture containing a drug which is pharmacologically or biologically effective when administered in a therapeutically effective amount. The term “agent” is also intended to have its broadest interpretation and is used to include any therapeutic agent or drug. The terms “drug”, “therapeutic agent”, “active agent” and “biologically active agent” are used interchangeably to refer to any therapeutically active substance that is delivered to a living organism to produce a desired, usually beneficial, effect.

The biologically active agents of the invention can also be in various forms, such as free bases, acids, charged or uncharged molecules, components of molecular complexes or nonirritating, pharmacologically acceptable salts. Further, simple derivatives of the active agents (such as ethers, esters, amides, etc.) which are easily hydrolyzed at body pH, enzymes, etc., can be employed.

It is to be understood that more than one biologically active agent may be incorporated into the coatings of this invention, and that the use of the term “active agent” in no way excludes the use of two or more such active agents or drugs.

The term “co-delivering”, as used herein, means that a supplemental agent(s) is administered transdermally either before the primary active agent is delivered, before and during transdermal flux of the active agent, during transdermal flux of the active agent, during and after transdermal flux of the active agent, and/or after transdermal flux of the active agent.

The term “microprojections”, as used herein, refers to piercing elements which are adapted to pierce or cut through the stratum corneum into the underlying epidermis layer, or epidermis and dermis layers, of the skin of a living animal, particularly, a mammal and, more particularly, a human.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 11. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. The active agent, if disposed therein, can be in solution or suspension in the formulation.

The term “biocompatible coating” and “solid coating”, as used herein, is meant to mean and include a “coating formulation” in a substantially solid state.

The term “vasoconstrictor”, as used herein, refers to a composition of matter or mixture that narrows the lumen of blood vessels and, hence, reduces peripheral blood flow. Examples of suitable vasoconstrictors include, without limitation, amidephrine, cafaminol, cyclopentamine, deoxyepinephrine, epinephrine, felypressin, indanazoline, metizoline, midodrine, naphazoline, nordefrin, octodrine, orinpressin, oxymetazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof.

The term “pathway patency modulator”, as used herein, refers to a composition of matter or mixture that slows the closure of pathways in the stratum corneum formed by the microprojections. Examples of suitable pathway patency modulators include, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

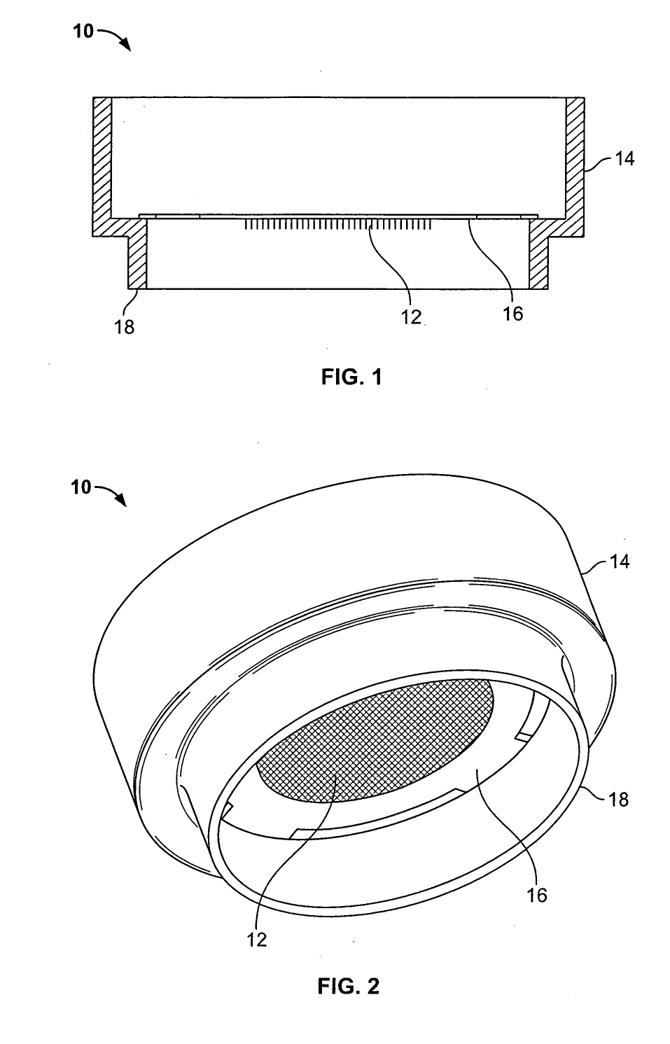

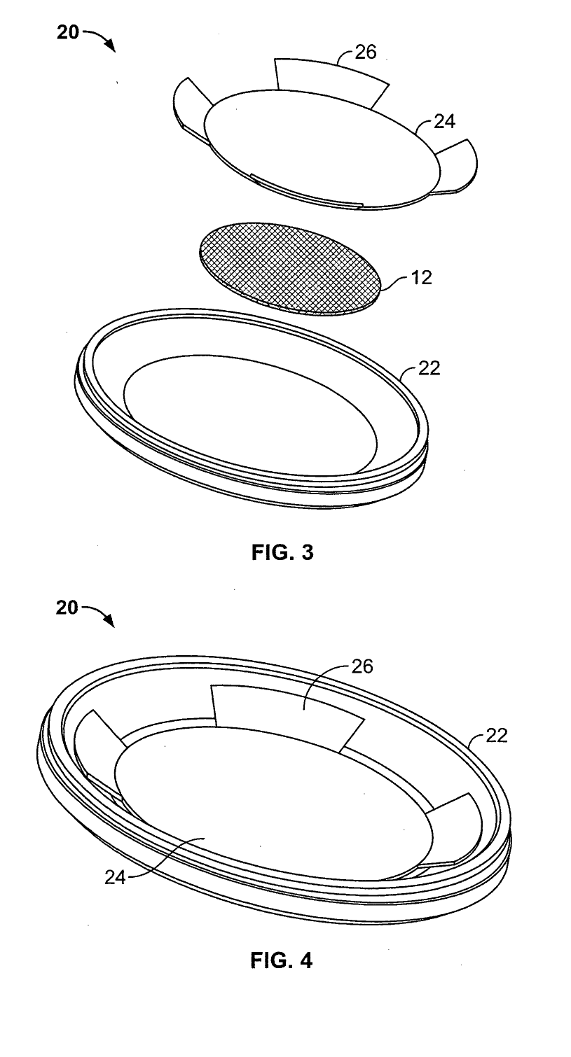

As discussed above, it is desirable to use an impact applicator to cause the microprojection member to pierce the stratum corneum of a patient in a uniform and reproducible manner. Accordingly, a prior art assembly 10 generally comprises a microprojection member 12 mounted in a prior art retainer 14 as shown in FIGS. 1 and 2. Preferably, the microprojection member 12 is suspended in prior art retainer ring 14 by frangible tabs of adhesive patch 16, as described in detail in U.S. Pat. No. 6,855,131, which is incorporated by reference herein in its entirety.

After placement of the microprojection member in the retainer ring 14, the microprojection member is applied to the patient's skin. Preferably, the microprojection member is applied to the patient's skin using an impact applicator, as described in Co-Pending U.S. application Ser. No. 09/976,978, which is incorporated by reference herein in its entirety.

As shown FIGS. 1 and 2, the configuration of prior art retainer 14 places microprojection member 12 in a recessed position. Since the microprojection member 12 is spaced away from a plane formed by end 18 of the retainer, it is difficult or impossible to apply a coating of a biologically active agent to microprojection member 12 once it is mounted on prior art retainer 14. Therefore, the microprojection member 12 must be mounted to prior art retainer 14 after application of the biologically active agent coating. In turn, this necessitates either aseptic manufacturing conditions during the steps of mounting the microprojection member 12 to the prior art retainer 14 or terminal sterilization, both of which are expensive and time consuming requirements. Further, sterilization of the microprojection member after it is coated with the agent risks degradation.

As indicated above, the present invention overcomes these drawbacks by providing an apparatus and method that permits a microprojection member to be mounted on a retainer and then coated with a biologically active agent. Since the microprojection member and retainer can be sterilized after they are assembled and prior to coating, this minimizes the number of manufacturing steps that must be carried out under aseptic conditions after the microprojection member is coated.

Accordingly, the transdermal delivery assembly of the present invention generally comprises a microprojection member having top and bottom surfaces and a plurality of stratum corneum-piercing microprojections that project from the bottom surface of the microprojection member and a retainer having first and second ends and a central opening wherein the microprojection member is secured to the retainer within the central opening and wherein the microprojection member is positioned adjacent the first end of the retainer so that at least a portion of the microprojections extend beyond a plane formed by the first end of the retainer.

Turning now to FIGS. 3 and 4, a transdermal delivery assembly 20 of the present invention is shown which generally includes a microprojection member 12, a retainer 22 and an adhesive patch 24. Microprojection member 12 is secured to the patch 24 by the adhesive and patch 24 is preferably sized to contact the patient's skin around the perimeter of microprojection member 12 to help retain the microprojection member in contact with the patient after application. Adhesive patch 24 preferably has tabs 26 for securing the microprojection member 12 within retainer 22. Tabs 26 are preferably frangible so that actuation of the applicator will release patch 24 from retainer 22. Also preferably, retainer 22 has a sloped rim to facilitate contact with the tabs 26.

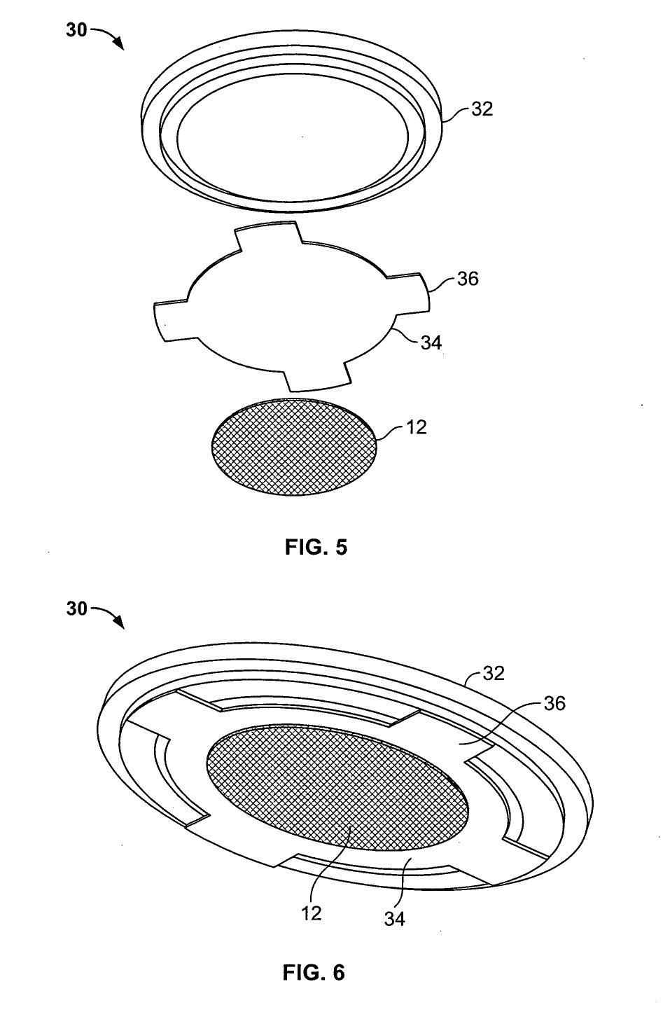

Another embodiment of the invention is shown in FIGS. 5 and 6, wherein the transdermal delivery assembly 30 also generally includes a microprojection member 12, a retainer 32 and an adhesive patch 34. In this embodiment, patch 34 has adhesive on one side for securing microprojection member 12 and retaining the patch on the patient's skin. A separate adhesive on the other side of patch 34 secures the patch to retainer 32. Preferably, the portion of patch 34 that is secured to retainer 32 is minimized and comprises tabs 36, that are also preferably frangible.

As can be seen, the retainers of the present invention position the microprojection member so that at least a portion of the microprojections extend beyond a plane formed by the end of the retainer. This configuration allows a biocompatible coating containing a biologically active agent to be applied to the microprojection member after it is mounted to the retainer.

According to the invention, the coating can be applied to the microprojection member by a variety of known methods. Preferably, the coating is only applied to those portions the microprojection member that pierce the skin.

A preferred coating method comprises roller coating, which employs a roller coating mechanism that similarly limits the coating to the tips of the microprojections. The roller coating method is disclosed in U.S. Pat. No. 6,855,372, which is incorporated by reference herein in its entirety. As discussed in detail in the noted patent, the disclosed roller coating method provides a smooth coating that is not easily dislodged from the microprojections during skin piercing.

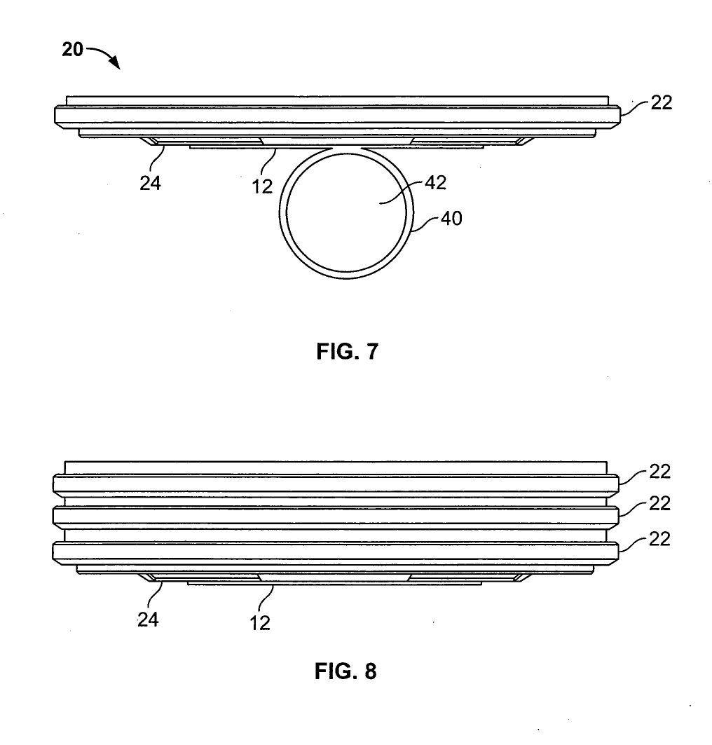

For example, FIG. 7 shows a coating of a biologically active agent formulation 40 being applied to the microprojection member 12 of transdermal delivery assembly 20 by a rotating drum 42. The position of microprojection member 12 within retainer 22 allows the microprojections to come into contact with a film of agent formulation 40 carried by drum 42 without interference from retainer 22.

Another coating method comprises dip-coating. Dip-coating can be described as a means to coat the microprojections by partially or totally immersing the microprojections into a coating formulation. By use of a partial immersion technique, it is possible to limit the coating to the tips of the microprojections. As can be appreciated, microprojection member 12 can be dipped into a reservoir of the coating formulation without contacting retainer 22 (or 32) with the coating formulation.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections and then dried.

Pattern coating can also be employed to coat the microprojections. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

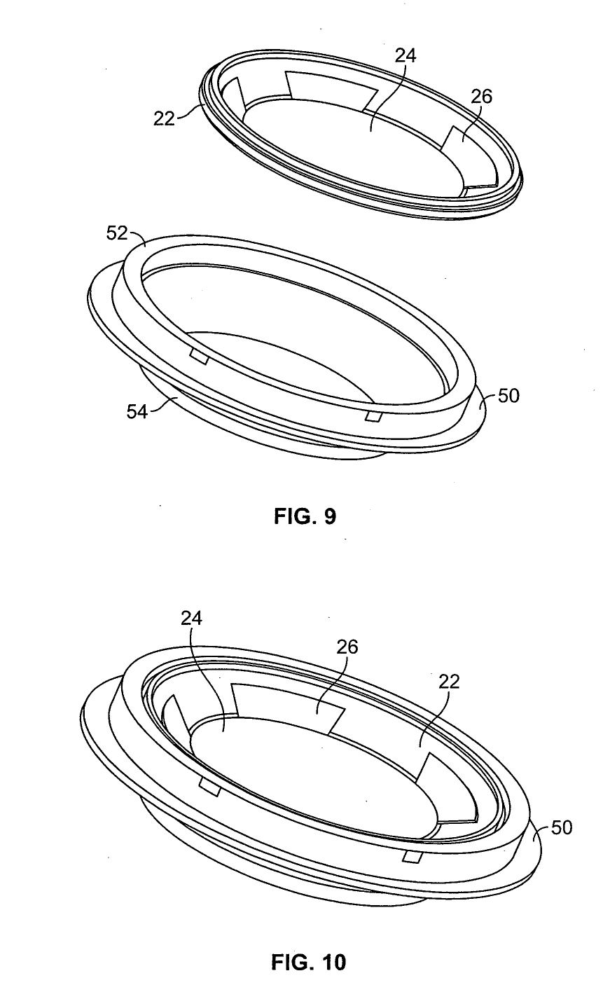

A further aspect of the invention allows multiple transdermal delivery assemblies 20 to be stacked as shown in FIG. 8. As can be appreciated, by positioning the microprojection member adjacent one end of retainer 22, a void is created at the opposing end. Preferably, retainer 22 (or 32) is configured to nest with like retainers as shown in FIG. 8. In this stacked configuration, microprojection member 12 and patch 24 are positioned within the void at the opposing end of the adjacent retainer, preventing the microprojection member 12 and patch 24 from coming into contact with the assembly of the adjacent retainer.

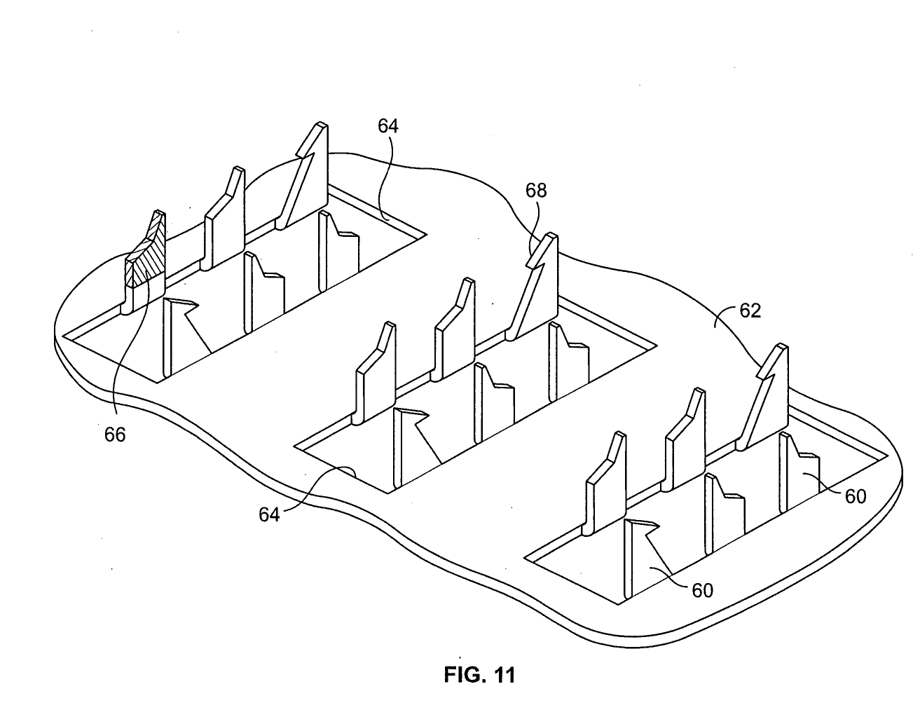

In another embodiment of the invention as shown in FIGS. 9 and 10, retainer 22 (or 32) is configured to mate with a housing 50. Housing 50 preferably has opposing ends, a first end 52 adapted to attach to an impact applicator and a second end 54 that contacts the patient's skin. Also preferably, housing 50 is configured to position retainer 22 so that inadvertent contact with microprojection member 12 is minimized by spacing retainer 22 away from each end. Although microprojection member 12 must already be coated with the active agent when retainer 22 is placed within housing 50, it is relatively easy to maintain aseptic conditions for this assembly step.

In the noted embodiments, the retainers 22 and 32 have been shown as being generally circular or ring shaped, however any suitable shape or configuration can be employed as desired so long as a central opening is defined within which the microprojection member can be secured and so that at least a portion of the microprojections extend beyond the plane formed by the end of the retainer.

Referring now to FIG. 11, there is shown a portion of one embodiment of a microprojection member 12 for use with the present invention. As illustrated, the microprojection member 12 includes an array of microprojections 60 that project from a sheet 62. The microprojections 60 preferably extend at substantially a 90° angle from the sheet 62, which in the noted embodiment includes openings 64. In this embodiment, the microprojections 60 are formed by etching or punching a plurality of microprojections 60 from a thin metal sheet 62 and bending the microprojections out of the plane of the sheet.

In one embodiment of the invention, the piercing elements have a projection length less than 1000 microns. In a further embodiment, the piercing elements have a projection length of less than 500 microns, more preferably, less than 250 microns. The microprojections further have a width in the range of approximately 25-500 microns and a thickness in the range of approximately 10-100 microns. The microprojections may be formed in different shapes, such as needles, blades, pins, punches, and combinations thereof.

In one embodiment of the invention, the microprojection member 12 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

To enhance the biocompatibility of the microprojection member 12 (e.g., to minimize bleeding and irritation following application to the skin of a subject), in a further embodiment, the microprojections 60 preferably have a length less than 145 μm, more preferably, in the range of approximately 50-145 μm, even more preferably, in the range of approximately 70-140 μm. Further, the microprojection member 12 comprises an array preferably having a microprojection density greater than 100 microprojections/cm², more preferably, in the range of approximately 200-3000 microprojections/cm².

The microprojection member 12 can be manufactured from various metals, such as stainless steel, titanium, nickel titanium alloys, or similar biocompatible materials.

According to the invention, the microprojection member 12 can also be constructed out of a non-conductive material, such as a polymer.

Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. Application Ser. No. 60/484,142, which is incorporated by reference herein.

Microprojection members that can be employed with the present invention include, but are not limited to, the members disclosed in U.S. Pat. Nos. 6,083,196, 6,050,988 and 6,091,975, 6,230,051 B1, 6,322,808 and Co-Pending U.S. application Ser. No. 10/045,842, which are incorporated by reference herein in their entirety which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the active agent to be delivered can be contained in a biocompatible coating 66 that is disposed on the microprojection member 12. The microprojections 60 can further include means adapted to receive and/or enhance the volume of the coating 66, such as apertures (not shown), grooves (not shown), surface irregularities (not shown) or similar modifications, wherein the means provides increased surface area upon which a greater amount of coating can be deposited. Further, the microprojections 60 can be formed with a hook or barb 68 configured to retain microprojection member 12 in contact with the patient's skin.

In certain embodiments of the invention, the biologically active agent comprises an agent active in one of the major therapeutic areas including, but not limited to: anti-infectives such as antibiotics and antiviral agents; analgesics, including fentanyl, sufentanil, remifentanil, buprenorphine and analgesic combinations; anesthetics; anorexics; antiarthritics; antiasthmatic agents such as terbutaline; anticonvulsants; antidepressants; antidiabetic agents; antidiarrheals; antihistamines; anti-inflammatory agents; antimigraine preparations; antimotion sickness preparations such as scopolamine and ondansetron; antinauseants; antineoplastics; antiparkinsonism drugs; antipruritics; antipsychotics; antipyretics; antispasmodics, including gastrointestinal and urinary; anticholinergics; sympathomimetrics; xanthine derivatives; cardiovascular preparations, including calcium channel blockers such as nifedipine; beta blockers; beta-agonists such as dobutamine and ritodrine; antiarrythmics; antihypertensives such as atenolol; ACE inhibitors such as ranitidine; diuretics; vasodilators, including general, coronary, peripheral, and cerebral; central nervous system stimulants; cough and cold preparations; decongestants; diagnostics; hormones such as parathyroid hormone; hypnotics; immunosuppressants; muscle relaxants; parasympatholytics; parasympathomimetrics; prostaglandins; proteins; peptides; psychostimulants; sedatives; and tranquilizers. Other suitable agents include vasoconstrictors, anti-healing agents and pathway patency modulators.

Further specific examples of agents include, without limitation, growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), amylin, insulinotropin, GLIP1, granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, alpha MSH, VEGF, PYY and hBNP.

Yet other suitable biologically active agents include immunologically active agents including, without limitation, viruses, bacteria, protein-based vaccines, polysaccharide-based vaccines, proteins, polysaccharide conjugates, oligosaccharides, lipoproteins, immunogenic materials, antigenic agents and vaccine adjuvants. Specific examples of vaccine delivery can be found in Co-Pending application Ser. Nos. 10/127,171 and 10/971,877, which are hereby incorporated in their entirety by reference.

Suitable immunologically active agents include, without limitation, antigens in the form of proteins, polysaccharide conjugates, oligosaccharides, and lipoproteins. Specific subunit vaccines include, without limitation, tetanus toxoid, diphtheria toxoid, botulinum toxoid, hemaglutinins, hepatitis B surface antigen, Bordetella pertussis (recombinant PT accince-acellular), Clostridium tetani (purified, recombinant), Corynebacterium diphtheria (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Suitable immune response augmenting adjuvants which, together with the antigen, can comprise the immunologically active agent include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK•HCl (IL-LS163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

As will be appreciated by one having ordinary skill in the art, with few exceptions, alum-adjuvanted vaccine formulations typically lose potency upon freezing and drying. To preserve the potency and/or immunogenicity of the alum-adsorbed vaccine formulations of the invention, the noted formulations can be further processed as disclosed in Provisional Application No. 60/649,275, filed Jan. 31, 2005; which is expressly incorporated by reference herein in its entirety.

Further details regarding these and other aspects of suitable coating formulations can be found in Co-Pending U.S. patent application Ser. Nos. 10/884,603, filed Jun. 29, 2004, and 11/034,891, filed Jan. 12, 2005, both of which are incorporated by reference herein in their entirety.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A transdermal delivery assembly, comprising:
a microprojection member having top and bottom surfaces and a plurality of stratum corneum-piercing microprojections that project from said bottom surface of said microprojection member; and
a retainer having first and second ends and a central opening;
wherein said microprojection member is secured to said retainer within said central opening and wherein said microprojection member is positioned adjacent said first end of said retainer so that at least a portion of said microprojections extend beyond a plane formed by said first end of said retainer.

2. The assembly of claim 1, further comprising an adhesive patch, wherein said microprojection member is secured to said patch and said patch is secured to said retainer.

3. The assembly of claim 2, wherein said patch is secured to said retainer by frangible tabs.

4. The assembly of claim 2, wherein said patch has first and second sides and wherein said microprojection member is secured to said first side.

5. The assembly of claim 4, wherein said patch is secured to said retainer by an adhesive on said first side.

6. The assembly of claim 4, wherein said patch is secured to said retainer by an adhesive on said second side.

7. The assembly of claim 2, wherein said first end of said retainer is configured to nest with said second end of said retainer so that a plurality of retainers can be stacked.

8. The assembly of claim 7, wherein said microprojection member is secured to said retainer so that said microprojection member does not contact adjacent microprojection members and adhesive patches when a plurality of assemblies are stacked.

9. The assembly of claim 1, further comprising a housing having first and second ends and a central opening, wherein said housing is adapted to receive and position said retainer within said central opening of said housing and wherein said retainer is disposed within said housing.

10. The assembly of claim 9, wherein said first end of said housing is adapted to releasably attach to an impact applicator.

11. The assembly of claim 9, wherein said retainer is positioned within said housing so that said microprojection member is spaced away from said first and second ends of said housing.

12. The assembly of claim 1, further comprising a biologically active agent disposed on said microprojections in a biocompatible coating.

13. The assembly of claim 12, wherein said active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones, hGH, HMG, desmopressin acetate, follicle luteoids, aANF, growth factors, growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), amylin, insulinotropin, GLIP1, granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs, ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, alpha MSH, VEGF, PYY and hBNP.

14. The assembly of claim 12, wherein said active agent is an immunologically active agent selected from the group consisting of proteins, polysaccharide conjugates, oligosaccharides, lipoproteins, tetanus toxoid, diphtheria toxoid, botulinum toxoid, hemaglutinins, hepatitis B surface antigen, Bordetella pertussis (recombinant PT accince-acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre 51, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

15. The assembly of claim 12, wherein said biocompatible coating further comprises at least one vasoconstrictor.

16. The assembly of claim 12, wherein said biocompatible coating further comprises at least one pathway patency modulator.

17. A method for producing a transdermal delivery assembly, comprising the steps of:
providing a microprojection member having top and bottom surfaces and a plurality of stratum corneum-piercing microprojections that project from said bottom surface of said microprojection member;
providing a retainer having first and second ends and a central opening; and
securing said microprojection member to said retainer within said central opening to form said transdermal delivery assembly wherein said microprojection-member is positioned adjacent said first end of said retainer so that at least a portion of said microprojections extend beyond a plane formed by said first end of said retainer.

18. The method of claim 17, further comprising the step of providing an adhesive patch, wherein the step of securing said microprojection member to said retainer comprises securing said microprojection member to said patch and securing said patch to said retainer.

19. The method of claim 18, further comprising the step of sterilizing said transdermal delivery assembly.

20. The method of claim 19, further comprising the step of applying a biocompatible coating containing at least one biologically active agent to said microprojection member after said transdermal delivery assembly is sterilized.

21. The method of claim 20, wherein said step of applying a biocompatible coating comprises roller coating.

22. The method of claim 20, wherein said step of applying a biocompatible coating comprises dip-coating.

23. The method of claim 20, further comprising the steps of providing a housing having first and second ends and a central opening, wherein said housing is adapted to receive and position said retainer within said central opening of said housing and placing said retainer within said housing after applying said biocompatible coating.
